(19) United States
(12) Patent Application Publication
(10) Pub. No.: US 2024/0325453 A1
(43) Pub. Date: Oct. 3, 2024

(54) ENGINEERED GLIAL CELLS FOR DESIGNER NEURAL REPAIR

(71) Applicants: Michael Aron Lane, Philadelphia, PA (US); Lyandysha Viktorovna Zholudeva, Concord, CA (US); Liang Qiang, Philadelphia, PA (US); Alessia Niceforo, Philadelphia, PA (US)

(72) Inventors: Michael Aron Lane, Philadelphia, PA (US); Lyandysha Viktorovna Zholudeva, Concord, CA (US); Liang Qiang, Philadelphia, PA (US); Alessia Niceforo, Philadelphia, PA (US)

(21) Appl. No.: 18/620,565

(22) Filed: Mar. 28, 2024

Related U.S. Application Data
(60) Provisional application No. 63/492,696, filed on Mar. 28, 2023.

(51) Int. Cl. A61K 35/30; A61P 25/00; C12N 5/079; C12N 15/63

(52) U.S. Cl. CPC A61K 35/30; A61P 25/00; C12N 5/0622; C12N 15/635; C12N 2510/00

(57) ABSTRACT

Compositions and methods for administering Doxycycline-activatable cells for repair of the nervous system after injury or disease are contemplated herein.

### PRIORITY

This application claims the benefit of U.S. Provisional Application No. 63/492,696, filed Mar. 28, 2023, the content of which is herein incorporated by reference in its entirety.

### BACKGROUND OF THE INVENTION

The nervous system is a complex highly specialized network that organizes explains, and direct interactions between the person and the world. The nervous system controls a variety of voluntary and involuntary functions. Accordingly, damage to the nervous system causes a variety of problems depending on the area that is damaged. Damage to the nervous system can occur slowly and cause a gradual loss of function or may be caused by a sudden and life-threatening problem. Symptoms are often severe; however, options for treating this damage are limited.

### SUMMARY OF THE INVENTION

In decades of transplantation work, many different types of cells have been transplanted without having control over them after they are transplant. Generally, glial and neuronal cells are administered in combination to promote the survival of the transplanted cells and promote regeneration and repair of the nervous system. Glial cells are often needed to support donor neuronal cells. They survive more easily and distribute more widely. As the cells are allowed to integrate into the nervous system without intervention, distinct pockets of glial cells can be found in patients after administration. These pockets can help support a limited degree of repair early after transplantation, but then they persist only as glial accumulations that likely have little-to-know function.

Furthermore, these techniques rely on spontaneous mechanisms of action from the donor cells to have a therapeutic effect (supporting early-stage tissue remodeling and growth). Surviving glial cells do not have ongoing reparative functions, and it has not been possible to transform these cells into functioning neurons, as can occur in more regenerative non-mammalian species (e.g., fish).

No other invention has been able to demonstrate direct conversion of cells that have been transplanted into a pathological environment. Thus, there is a need for safe and effective pharmacologic treatments capable of transplanting and activating cells for the repair of an injured or diseased nervous system.

The present invention comprises engineered glial cells comprising an exogenous nucleic acid comprising a transcription factor under the control of a regulatable promoter. In certain embodiments, the regulatable promoter is a doxycycline (DOX)-inducible expression control system. In certain embodiments, the transcription factor comprises one or more of VSX2 (also called HOX10), Ascl1, miR124, and/or miR9/9*.

In another aspect, the present invention comprises methods for repairing a damaged nervous system in a subject in need thereof, the method comprising administering to the subject the cells of any one of the preceding claims and activating the response of said cells, thereby inducing reprogramming of the cells into neurons. In certain embodiments, the damage to the nervous system is caused by injury or disease. In certain embodiments, said response is activated by administration of DOX to the subject. In certain embodiments, the response is activated weeks or months after administration.

In certain embodiments the patient has damage to at least one of the brain or the spinal cord. In certain embodiments, the unmodified cells are obtained from at least one of the patient or a donor and used to generate the engineered cells. In certain embodiments, the unmodified cells are human or non-human PCS. In another aspect, the unmodified cells are stem cells and are differentiated to glial cells. In yet another aspect, administration of the cells improves motor recovery of the patient when compared to an untreated control. In certain embodiments, the cells display at least one of anatomical and functional evidence of connectivity.

In one aspect, the cells are administered prior to surgical treatment. In another aspect, the cells are administered after surgical treatment. In certain embodiments, the present invention further comprising said patient participating in rehabilitative physical therapy. In certain embodiments, the rehabilitative physical therapy maintains or strengthens muscle function or redevelops fine motor skills.

Still other aspects and advantages of these compositions and methods are described further in the following detailed description of the preferred embodiments thereof.

### BRIEF DESCRIPTION OF THE FIGURES

FIGS. 1A-1B. Vector Map for DOX-inducible constructs used to infect cells (FIG. 1A). Vector components for DOX-inducible construct used to infect cells (FIG. 1B).

FIGS. 2A-2E. Schematic of the cervical spinal cord and phrenic network (FIG. 2A). Cross-sections of the adult rat intact (FIG. 2B) and injured (laterally contused) cervical spinal cords (FIG. 2C). Diaphragm electromyograms (EMGs) from uninjured (FIG. 2D) and contused animals (FIG. 2E; 12 weeks post-injury).

FIGS. 3A-3E. Schematic diagram of the general procedure used to obtain glial restricted progenitor cells (GRPs) that are transplanted into the lesion cavity acutely (1 week) or chronically (1 month) post-SCI. (FIG. 3A) Developing spinal cord tissue from GFP expressing E13.5 rat pups was dissected out, mechanically and enzymatically dissociated and (FIG. 3B) cultured for three days to reach confluency, and frozen, to generate stocks. Separate samples of cells are cultured with lenti-virus to allow for doxycycline-induced gene regulation and direct reprogramming of glial cells into neurons. (FIG. 3C) These cells are then thawed, plated, and stereotaxically injected into (FIG. 3D) the lesion cavity. Animals recovered for 4 wks, and then a subset will be given doxycycline (FIG. 3E).

FIGS. 4A-4H. Principle component analysis (PCA) was used to compare RNA isolated from developing (E14, 15; P3, 6) spinal cords (FIG. 4A) and cultured GRPs and NPCs compared to E14 spinal cords (FIG. 4E). Heatmap comparing the expression of pan-astrocytic genes and genes relevant to either more “inflammatory” (Inflam.) and or “scar forming or proliferative” (Scar/Prolif.) glial phenotypes between embryonic (E14,15) and postnatal (P3,6) spinal cords (FIG. 4B), with specific examples of genes in (FIG. 4C) and (FIG. 4D). Similar analysis across the cultured cells (GRPs and NPCs, compared to E14 spinal cord) is represented as a heatmap in (FIG. 4F), with specific examples of genes in (FIG. 4G) and (FIG. 4H). E14,15: Embryonic Day 14,15; P3,6: Postnatal Day 3,6; GRP-GFP: glial restricted progenitors with GFP reporter; NPC: neuronal and glial restricted progenitors.

FIGS. 5A-5G. Cultured glial restricted progenitors (GRPs) were transplanted 1 week post injury at two doses: 500,000 cells/animal (FIG. 5A) and 1 million cells/animal (FIG. 5B). Surviving GRPs (FIG. 5C) were observed to express GFAP (FIG. 5D) and guide sprouting 5HT axons (FIG. 5E) into and through the injury site. Overlay in (FIG. 5F). (FIG. 5G) Amplitude of diaphragm contraction measured with electromyography was used to assess recovery 4 weeks after transplantation (green bar, 500,000 cells/animal) compared to vehicle-recipients (blue bar) under baseline and hypoxic challenge. Data points in scatterplot represent individual animals. Scale bar is 1 mm in (FIGS. 5A-5B) and 200 microns in (FIGS. 5C-5F).

FIGS. 6A-6D. Schematic representation of timeline of conversion from primary astrocytes to neurons. (FIG. 5A) Phase images in (FIG. 6B) illustrate the progressive astrocyte to neuron reprograming. Immunocytochemistry was used to confirm glial derived neurons (MAP2+, green; TauR1+, red), with decreasing expression of GFAP (white) with DAPI (blue) (FIG. 6C). Overlay is shown in (FIG. 6D). Scale bar is 80 (FIGS. 6B, 6C) and 250 microns (FIG. 6D).

FIG. 7. Immunofluorescence of primary astrocyte cultures generated from postnatal days 2-3 (P2-3) rat cortex. (GAFP, white; DAPI; blue) Scale bar 500 microns.

FIG. 8. A schematic of the experimental timeline showing the intended conversion of primary astrocytes to neurons.

FIGS. 9A-9J. Phase images to investigate changes in cell morphology during conversion of astrocytes. Days-2 through 0 display an undifferentiated astrocytic morphology (FIG. 9A). rtT A, Ascl1, miRs at Day 1 (FIGS. 9B, 9E, and 9H) rtTA, Ascl1, miRs at Day 5 (FIGS. 9C, 9F, and 9I). rtT A, Ascl1, miRs at Day 15 (FIGS. 9D, 9G, and 9J) Scale bar: 250 microns in FIGS. 9A-9J.

FIGS. 10A-10O. Immunocytochemistry for astrocytic marker GFAP (white), neuronal nuclear protein NeuN (green) and neuronal cytoskeletal marker β3-tubulin (red were used to assess protein expression in vitro. (FIGS. 10A-10L) Magnification of FIGS. 10D, 10H, and 10L are provided as FIGS. 10M, 10N, and 100 respectively. Scale bar: 100 microns for FIGS. 10A-10L. Scale bar: 500 microns for FIGS. 10M-10O.

FIGS. 11A-11F. Phase images of astrocytes-to-neurons replated in MEA single well at day 15, with the tree different conditions: ItTA, Ascl1, and miRs, respectively. (FIGS. 11A-11C). Neuronal spikes are absent from the cells plated with rtTA (FIG. 11D) but present in cells plated with Ascl1 (FIG. 11E) or miRs (FIG. 11F). Scale bar: 250 microns in FIGS. 11A-11C.

### DETAILED DESCRIPTION OF THE INVENTION

In certain embodiments of this invention, the use of doxycycline (DOX)-activatable (or other inducible promoter) cells (e.g., glial cells) for repair of the nervous system (e.g., brain and spinal cord) after injury or disease are described. DOX-activatable cells refers to cells that are responsive to treatment with doxycycline. A novel strategy for neural repair that involves transplanting donor glial cells that can be reprogrammed in the presence of, but not limited to, DOX. is disclosed herein. Doing so means that the cells can be transplanted as glial cells, survive the inhibitory environment known to comprise the injured central nervous system, then weeks to months after transplantation, the cells can reprogrammed to undergo directed differentiation with administration of DOX, to reprogram the transplanted glial cells directly into neurons.

Emerging technologies, such as cell-based repair strategies, offer new promise for some of the most devastating medical conditions that currently lack treatments. However, to harness the full therapeutic potential of stem cells, it will be necessary to understand how to direct their differentiation to appropriate cell phenotypes and ensure that their phenotype and function persist after transplantation into a pathologic environment. Using a model of an acutely and chronically injured spinal cord, the inventors tested the therapeutic potential of transplanted DOX-activatable glial cells.

This invention and the use of it to repair the injured or diseased central nervous system gives precise control over when the transplanted cells are active and/or silenced, by providing control over the transplanted cells themselves.

In certain embodiments, the invention is a biological product intended for transplantation into the injured or diseased nervous system that can be converted from glial cells directly into neurons when exposed to DOX. This ability enables the entrainment of transplanted cells to perform in a functionally and therapeutically relevant way. These cells are a specific type of glial cells that are engineered to be relevant to neural repair.

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, where compositions, components, methods, or steps are described as required in one or more embodiments, additional embodiments are contemplated and are disclosed hereby for fewer compositions, components, methods, or steps, and for fewer compositions, components, methods, or steps in addition to other compositions, components, methods, or steps. All compositions, components, methods, or steps provided herein may be combined with one or more of any of the other compositions, components, methods, or steps provided herein unless otherwise indicated.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

A “sample” refers to a sample from a subject that may be tested. The sample may comprise cells, and it may comprise body fluids, such as blood, serum, plasma, cerebral spinal fluid, urine, saliva, tears, pleural fluid, and the like.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys. The term does not denote a particular age or sex. Thus, adult and newborn subjects, as well as fetuses, whether male or female, are intended to be covered.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

The term “pharmaceutically acceptable carrier” or “diluent” is intended to include any and all solvents, dispersion media, coatings, antibacterial and antifungal agents, isotonic and absorption delaying agents, adjuvants and the like, compatible with administration to humans. In one embodiment, the diluent is saline or buffered saline.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, ameliorization, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

“Human pluripotent stem cells” or “hPSCs”, which include human embryonic stem cells (hESCs) and human induced pluripotent stem cells (hiPSCs), are cells that can self-renew indefinitely in culture while maintaining the ability to become almost any cell type in the human body. In certain embodiments, the hPSCs are genetically altered to respond DOX administration. These cells are then differentiated into different cells in the central nervous system (CNS).

“Neural progenitor cells (NPCs)” are the progenitor cells of the CNS that give rise to many, if not all, of the glial and neuronal cell types that populate the CNS. NPCs do not generate the non-neural cells that are also present in the CNS, such as immune system cells. NPCs are present in the CNS of developing embryos but are also found in the neonatal and mature adult brain, and therefore are not strictly embryonic stem cells. NPCs are characterized based on their location in the brain, morphology, gene expression profile, temporal distribution and function. In general, embryonic NPCs have more potential than NPCs in the adult brain. In certain embodiments, the NPC is a genetically modified glial cell.

Neurons, which process information, and glial cells (or neuroglia), which provide mechanical and metabolic support to the nervous system and modulate information processed by neurons, are the two main classes of cells of the central nervous system. Non-limiting examples of neurons include sensory neurons (also referred to as afferent neurons) transfer information from the external environment to the central nervous system, motor neurons (also referred to as efferent neurons) transfer information from the central nervous system to the external environment, and interneurons (also referred to as association neurons) process information in the central nervous system and transfers the information from one neuron to the other within the central nervous system. CNS progenitor cells can be neural progenitor cells or glial progenitor cells.

“Glial cells” provide physical and chemical support to neurons and help maintain their environment. There are five types of glial cells in the CNS including astrocytes, oligodendrocytes, microglia, ependymal cells, and radial glia. In the peripheral nervous system (PNS) there are two types of glial cells, Schwann cells and satellite cells. Each type of glial cell can be further differentiated into additional subtypes. For example, astrocytes include both protoplasmic and fibrous astrocytes. Although glial cells generally work to support the neurons, each glial cell type has distinct jobs.

The term “doxycycline” or “DOX” refers to a broad-spectrum tetracycline class antibiotic having the chemical formula C₂₂H₂₄N₂O₈. DOX can be administered to specially designed cells to allow researchers to quickly control cellular switches. After administration, DOX has rapid clearance which allows for more precise control over the pathway and easy repetition as required. Additionally, administration of DOX can precisely manipulate a large population of the modified cells using multiple injections. In view of these, DOX allows for more controlled activation of these cells than other available techniques.

Also contemplated herein, other tetracycline class antibiotics can be administered to the specially designed cells in the place of doxycycline. Tetracyclines are classified as broad-spectrum protein synthesis inhibitors. Naturally occurring drugs in this class are tetracycline, chlortetracycline, oxytetracycline, and demeclocycline. Other tetracyclines include, without limitation, lymecycline, methacycline, minocycline, rolitetracycline, tigecycline, ervacycline, sarecycline, and omadacycline. Tetracyclines can be administered orally, topically, intramuscularly or intravenously.

The nervous system is the major controlling, regulating, and communication system in the body. It controls complicated processes like movement, thought and memory and plays an essential role in natural bodily processes. The nervous system affects every aspect of health including, without limitation, thoughts, memory, learning, feelings, movements, balance, coordination, senses, including sight, hearing, taste, touch and feeling, sleep, healing, aging, heartbeat, breathing patterns, stress responses, digestion, and other body processes. The nervous system uses specialized cells called neurons to send signals throughout the body. These signals carry data to and from the brain.

A “damaged nervous system” refers to any disorder or condition that causes an injured nerve. “Injured nerves” refer to any nerve that has trouble sending or receiving a signal. In certain embodiments, the nerves are so damaged that they are completely unable to send or receive signals. Nerve injury can cause numbness, pain, inability to move, and/or death. Other symptoms include, vision problems, headaches, slurred speech, loss of sensation, tremors or tics (random muscle movements), changes in behavior or memory, problems with coordination or movement, and muscle weakness.

The nervous system can be damaged by disease, such as cancers, autoimmune diseases like diabetes, lupus and rheumatoid arthritis, multiple sclerosis, stroke, accidental injury, pressure on the nerve, toxic chemicals including certain medicines, and advanced age. In certain circumstances, surgery is required to rectify the damage to the nervous system.

The phrase “TeSR media” refers to any medium that can be used to maintain hPSC cultures. Adjustments to a TeSR media will cause differentiation of the hPSC.

The phrase “CHIR” refers to the GSK3β inhibitor CHIR99021, 6-[[2-[[4-(2,4-Dichlorophenyl)-5-(5-methyl-1H-imidazol-2-yl)-2-pyrimidinyl]amino]ethyl]amino]-3-pyridinecarbonitrile. CHIR99021 is an aminopyrimidine derivative. CHIR99021 promotes self-renewal potential of embryonic stem cells (ESCs) of mice by inhibiting glycogen synthase kinase-3 (GSK-3) activity and potentiates the upregulation of β-catenin and c-Myc functions. CHIR99021 promotes self-renewal of ESCs by modulating transforming growth factor β (TGF-β), Notch and mitogen-activated protein kinases (MAPK) signaling pathways. CHIR99021 is an agonist of wingless/integrated (wnt) signaling, upregulates cyclinA expression and promotes cell proliferation in non-small-cell lung cancer (NSCLC) cell lines.

SMADs comprise a family of structurally similar proteins that are the main single transducers for receptors of the TFG-B superfamily, which are critically important for regulating cell development and growth. An “SMAD inhibitor” reduces or decreases the activity or expression of SMAD. Dual SMAD inhibition refers to a process using SMAD inhibitors to rapidly differentiate hPSCs into early neurectoderm.

“Neural Induction Media” refers to Gibco® PSC Neural Induction Medium or other similar commercially available serum-free medium that provides high efficiency neural induction of human pluripotent stems cells (PSCs).

A Notch Pathway is a highly conserved signaling pathway that is crucial in development and is implicated in malignant transformation. A Notch Pathway inhibitor reduces or decreases the activity of the Notch Pathway. In certain embodiments, the Notch Pathway inhibitor is DAPT.

“BrainPhys Media” refers to the neuronal medium developed by Bardy et al (BrainPhys supports neurophysiological activity, Bardy et al, Proceedings of the National Academy of Sciences May 2015, 112 (20) E2725-E2734; DOI: 10.1073/pnas. 1504393112) and available from Stemcell Technologies (cat #ST-05790). BrainPhys Media simulates various in vivo conditions including inorganic salt concentration, glucose levels, and osmolarity. Additionally, BrainPhys Media allows for several Neuronal functions including spontaneous and evoked action potentials, spontaneous network calcium dynamics, excitatory synaptic activity, and inhibitory synaptic activity. To sustain cell survival and/or neural differentiation in vitro, supplements such as antioxidants, growth factors, hormones, and proteins can be added to basal media.

“G/BDNF” refers to brain derived neurotropic factor.

“IGF” refers to interferon gamma.

“CNTF” refers to Ciliary Neurotrophic Factor.

### Reprogrammable Glial Cells

The cells provided herein are modified such that they are able to be reprogrammed to neuronal cells by a controlled stimulus, at a desirable time. In one embodiment, the glial cells contain one or more transcription factors under the control of an inducible promoter, such as a doxycycline-inducible expression control system. In other embodiments, the glial cells contain a different inducible expression system, such as that have been develop for altering gene expression in bacteria and plant and animal cells.

In one embodiment, the doxycycline-inducible expression control system is a tetracycline-Controlled Operator system (Tet-On system; tetracycline-controlled operator system). The Tet-On system employs nucleic acid encoding a reverse tetracycline transactivator (rtTA) protein, which is a fusion of the tetracycline repressor (TetR) protein mutated at four amino acid positions to reverse the response to tetracycline/doxycycline, and the activation domain of VP16. In the absence of tetracycline (or a derivative thereof such as doxycycline) rtTA does not bind to TetO operator sequences and the polypeptide is not expressed. In the presence of tetracycline/doxycycline, rtTA binds to TetO sequences in the TRE and activates transcription of the nucleic acid downstream of the promoter. The Tet-On system is used herein activates specific proteins that differentiate glial cells into neuronal cells. These proteins include, without limitation, Ascl1 and a combination of miR124, miR9/9* (hereinafter, “miRs”).

Tet-On systems are described in Das et al., Curr Gene Ther. (2016) 16 (3): 156-67 (hereby incorporated by reference in its entirety) and include systems using 13 optimized rtTA variants such as the Tet-On Advanced system (which uses the rtTA variant protein rtTA2.sup.s-M2) and Tet-On 3G system.

The Tet-On Advanced systems are also described in Urlinger et al. Proc. Natl. Acad. Sci. U.S.A. (2000) 97 (14): 7963-8 (hereby incorporated by reference in entirety), and Kallunki T, et al. How to Choose the Right Inducible Gene Expression System for Mammalian Studies? Cells. 2019; 8 (8): 796. Tet-On 3G is described in Zhou et al., Gene Ther. 13 (19): 1382-1390 (hereby incorporated by reference in entirety).

Other inducible systems can also be used, such as cumate-controlled operator system, riboswitch-regulatable expression system, alone in combination with the systems described herein (such as tetracycline/cumate-controlled operator systems). These systems are commercially available, see for example, Kallunki et al. Cells. 2019 August; 8 (8): 796, in particular Table 3 (the reference is incorporated fully by reference herein).

The Inducible systems used herein activate specific proteins that promote differentiation of glial cells into neuronal cells. In certain embodiments, these proteins are transcription factors. These proteins include, without limitation, Vsx2 (also called Chx10), Ascl1, and a combination of miR124, miR9/9* (hereinafter, “miRs”), NeuroD1, Sox2.

Ascl1 is a transcription factor that plays a role in the neuronal commitment and differentiation and in the generation of olfactory and autonomic neurons. Ascl1 is central to the differentiation of the neuroblasts.

miR124 and miR9/9* are short non-coding RNA that are involved in post-transcriptional regulation of gene expression in multicellular organisms by affecting both the stability and translation of mRNAs. miR124 and miR9/9* are upregulated during neurodevelopment and adult neurogenesis.

Visual system homeobox 2 (VSX2) mediates differentiation of V2a interneurons by repression of motor neuron gene transcription, via competitively binding to response elements that are activated by the ISL1-LHX3 complex, such as VSX1 and MNX1

NeuroD1, or neurogenic differentiation 1 (also called beta-2) is a neural transcription factor. It is involved with cortical layer development in the developing brain and is considered a master regulator that results in downstream activation of additional transcription factors.

Sox2 is a transcription factor that plays a critical role in the maintenance of embryonic and neural stem cells. Activation in neural stem cells is crucial for the differentiation of cells into neuroectoderm.

### Vectors for Modifying Glial Cells

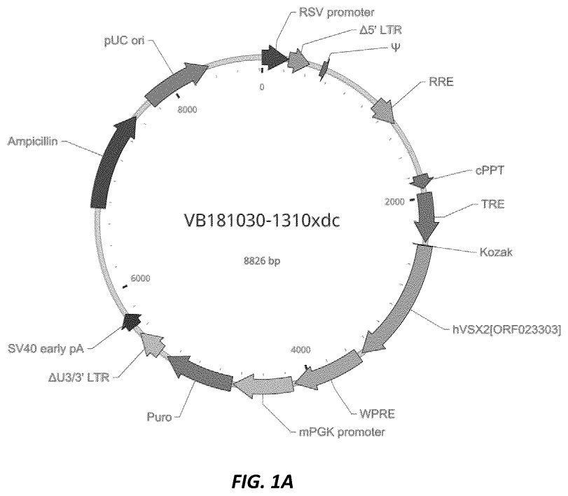

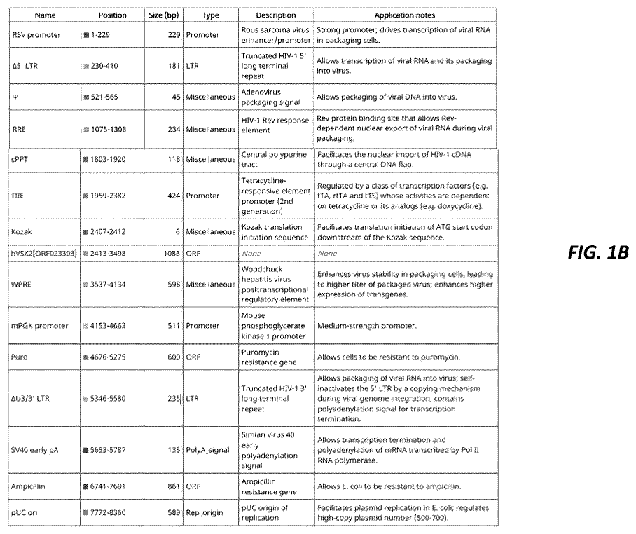

In certain embodiments, the cells are modified using a vector. As used herein, a “vector” comprises any genetic element including, without limitation, naked DNA, a phage, transposon, cosmid, episome, plasmid, bacteria, or a virus, which expresses, or causes to be expressed, a desired nucleic acid construct. An exemplary construct used to generate a vector is shown in FIG. 1.

In one embodiment, the vector is a non-pathogenic virus. In another embodiment, the vector is a non-replicating virus. In one embodiment, a desirable viral vector may be a retroviral vector, such as a lentiviral vector. In another embodiment, a desirable vector is an adenoviral vector. In still another embodiment, a suitable vector is an adeno-associated viral vector. Adeno, adeno-associated and lentiviruses are generally preferred because they infect actively dividing as well as resting and differentiated cells such as the stem cells, macrophages and neurons. A variety of adenovirus, lentivirus and AAV strains are available from the American Type Culture Collection, Manassas, Va., or available by request from a variety of commercial and institutional sources. Further, the sequences of many such strains are available from a variety of databases including, e.g., PubMed and GenBank.

In one embodiment, a lentiviral vector is used. Among useful vectors are the equine infectious anemia virus and feline as well as bovine immunodeficiency virus, and HIV-based vectors. A variety of useful lentivirus vectors, as well as the methods and manipulations for generating such vectors for use in transducing cells and expressing heterologous genes, e.g., N Manjunath et al, 2009 Adv Drug Deliv Rev., 61 (9): 732-745; Porter et al., N Engl J Med. 2011 Aug. 25; 365 (8): 725-33), among others.

In another embodiment, the vector used herein is an adenovirus vector. Such vectors can be constructed using adenovirus DNA of one or more of any of the known adenovirus serotypes. See, e.g., T. Shenk et al., Adenoviridae: The Viruses and their Replication”, Ch. 67, in FIELD'S VIROLOGY, 6.sup.th Ed., edited by B. N Fields et al, (Lippincott Raven Publishers, Philadelphia, 1996), p. 111-2112; U.S. Pat. No. 6,083,716, which describes the genome of two chimpanzee adenoviruses; U.S. Pat. No. 7,247,472; WO 2005/1071093, etc. One of skill in the art can readily construct a suitable adenovirus vector to carry and express a nucleotide construct as described herein. In another embodiment, the vector used herein is an adeno-associated virus (AAV) vector. Such vectors can be constructed using AAV DNA of one or more of the known AAV serotypes. Sec, e.g., U.S. Pat. Nos. 7,803,611; 7,696,179, among others.

In certain embodiments, the construct is delivered via a lipid nanoparticle. The term “lipid nanoparticle” refers to a lipid composition having a typically spherical structure with an average diameter of 10 to 1000 nanometers, e.g., 75 nm to 750 nm, or 100 nm and 350 nm, or between 250 nm to about 500 nm. In some formulations, lipid nanoparticles can comprise at least one cationic lipid, at least one noncationic lipid, and at least one conjugated lipid. Lipid nanoparticles known in the art that are suitable for encapsulating nucleic acids, such as mRNA, may be used. “Average diameter” is the average size of the population of nanoparticles comprising the lipophilic phase and the hydrophilic phase. The mean size of these systems can be measured by standard methods known by the person skilled in the art. Examples of suitable lipid nanoparticles for gene therapy is described, e.g., L. Battaglia and E. Ugazio, J Nanomaterials, Vol 2019, Article ID 283441, pp. 1-22; US2012/0183589A1; and WO 2012/170930 which are incorporated herein by reference in their entirety.

These vectors also include conventional control elements that permit transcription, translation and/or expression of the nucleic acid constructs in a cell transfected with the plasmid vector or infected with the viral vector. A great number of expression control sequences, including promoters which are native, constitutive, inducible and/or tissue-specific, are known in the art and may be utilized. In one embodiment, the promoter is selected based on the chosen vector. In another embodiment, when the vector is lentivirus, the promoter is RSV, U6, H1, CMV IE, EF-1α, ubiquitin C, or phosphoglycero-kinase (PGK) promoter. In one embodiment, the promoter is an RSV promoter. In another embodiment, when the vector is an AAV, the promoter is an RSV, U6, or CMV promoter. In another embodiment, when the vector is an adenovirus, the promoter is RSV, U6, CMV, or H1 promoters. In another embodiment, when the vector is Listeria monocytogenes, the promoter is a hly or actA promoter. Still other conventional expression control sequences include selectable markers or reporter genes, which may include sequences encoding geneticin, hygromycin, ampicillin or puromycin resistance, among others. Other components of the vector may include an origin of replication. Selection of these and other promoters and vector elements are conventional and many such sequences are available.

These vectors are generated using the techniques and sequences provided herein, in conjunction with techniques known to those of skill in the art. Such techniques include conventional cloning techniques of cDNA such as those described in texts (Sambrook et al, Molecular Cloning: A Laboratory Manual, Cold Spring Harbor Press, Cold Spring Harbor, N.Y.), use of overlapping oligonucleotide sequences, polymerase chain reaction, and any suitable method which provides the desired nucleotide sequence.

Thus, in one embodiment, using the information taught herein and publicly available and known vector construction components and techniques, one of skill in the art can construct a viral vector (or plasmid) that expresses the desired nucleic acid construct.

It should be understood that the compositions in the vector described herein are intended to be applied to other compositions, regiments, aspects, embodiments and methods described across the Specification.

### Methods of Generating Inducible Glial Cells

Provided herein are methods for generating e.g., DOX-activatable glial cells for use in the treatment of the nervous system in a subject in need thereof. By DOX-activatable it is meant that the cells engineered by this method respond to a stimulus that induces expression of one or more factors that results in reprogramming of the glial cell into a neuron. In certain embodiments, this stimulus is a tetracycline class antibiotic, e.g., DOX. In certain embodiments, the response to this stimulus can be activated or silenced at will. In certain embodiments, the cells are stimulated after several weeks or months after implantation into a subject.

In certain embodiments, the methods disclosed herein comprise generating glial cells from human pluripotent stem cells (hPSCs) obtained from a donor. In certain embodiments the donor is the same person that is treated using the method disclosed herein. In other embodiments, the donor is a healthy subject.

In certain embodiments, the hPSCs are first differentiated to neural progenitor cells. Methods of generating neural progenitors from hPSCs are known in the art. For example, see Walsh et al, Defined Culture Conditions Accelerate Small-molecule-assisted Neural Induction for the Production of Neural Progenitors from Human-induced Pluripotent Stem Cells, Cell Transportation, Feb. 2, 2018, 26 (12): 1890-1902, which is incorporated herein by reference. For example, to produce the neural progenitor cells, the cells obtained from the donor are plated on mTeSR media and contacted with (CHIR) and at least one Dual SMAD inhibitor (SMADi). In certain embodiments, the SMADi is selected from LEFTY1, Noggin and SB431542. In other embodiments, the SMAD pathway is inhibited using more than one agent, such as SB431542 and dorsomorphin (DM). The cells are then incubated for about 5 days. In certain embodiments, the cells are incubated for about 1-10 days. Further ranges/subranges, and integers within those ranges/subranges, are also contemplated, including but not limited to about 1-5 days, about 1-6 days, about 1-7 days, about 1-8 days, about 1-9 days, about 5-10 days, about 5-9 days, about 5-8 days, about 5-7 days, about 5-6 days, about 3-7 days, about 4-6 days, at least 2 days, at least 3 days, at least 4 days, at least 5 days, at least 6 days, at least 7 days, at least 8 days, at least 9 days, at least 10 days.

The cells are then replated in Neural Induction Medium. Such neural induction media are known in the art, e.g., DMEM/F12, 1% N2 supplement (Thermo Fisher Scientific), 2 μg ml—1 heparin (Sigma-Aldrich) and 1% penicillin/streptomycin. The CHIR and SMADi remain present in the Neural Induction Media and the cells are further contacted with Retinoic Acid (RA). The cells are incubated on this media for about 2 days, then the CHIR and SMADi are removed, and the cells are contacted with RA, a Notch Pathway Inhibitor, and purmorphamine (Pur). In certain embodiments the Notch Pathway inhibitor is DAPT. The cells are then allowed to incubate for about 10 days before being re-plated on BrainPhys Media (available from Stem Cell Technologies (cat #ST-05790)) before being administered to the patient. In certain embodiments, while on the BrainPhysMedia, the cells are contacted with G/BDNF, IGF, and CNTF.

In certain embodiments, the cells are replated on BrainPhys media after about 5-15 days. Further ranges/subranges, and integers within those ranges/subranges, are also contemplated, including but not limited to about 5-10 days, about 6-10 days, about 7-10 days, about 8-10 days, about 9-10 days, about 10-15 days, about 11-15 days, about 12-15 days, about 13-15 days, about 14-15 days, about 8-12 days, about 9-11 days, at least 2 days, at least 3 days, at least 4 days, at least 5 days, at least 6 days, at least 7 days, at least 8 days, at least 9 days, at least 10 days.

In certain embodiments, the glial cells are produced directly from the hPSCs. Many methods are known for producing glial cells from hPSCs.

The person of skill in the art can produce cells according to the methods taught herein, and techniques known in the art, e.g., Zheng, W. et al. “Differentiation of Glial Cells From hiPSCs: Potential Applications in Neurological Diseases and Cell Replacement Therapy” Frontiers in Cellular Neuroscience vol. 12, Art. 239 (August 2018); Leng K et al., “CRISPRi screens in human iPSC-derived astrocytes elucidate regulators of distinct inflammatory reactive states.” Nat Neurosci. 2022 November;25 (11): 1528-1542; and Tew J., et al., “An Efficient Platform for Astrocyte Differentiation from Human Induced Pluripotent Stem Cells.” Stem Cell Reports. 2017 Aug. 8; 9 (2): 600-614, each of which is incorporated herein by reference.

In one aspect, provided herein, is a vector comprising an engineered nucleic acid sequence encoding one or more factors under the control of an expression control system that is delivered to the target cells before, during, or after differentiation into glial cells. In certain embodiments, the vector is delivered to the target cell in vitro.

As used herein, the term “target cell” refers to any target cell in which expression of the expression control system is desired. In one embodiment, the target cell is an hPSC. In another embodiment, the target cell is a glial cell. In another embodiment, the target cell is another human cell that may be differentiated into a neuron.

Vectors useful in are described at length above. In certain embodiments, the vector is a synthetic or artificial viral particle in which an expression cassette containing a nucleic acid sequence encoding the expression control system is packaged in a viral capsid or envelope, where any viral genomic sequences also packaged within the viral capsid or envelope are replication-deficient, i.e., they cannot generate progeny virions but retain the ability to infect target cells. The viral vector can be selected from a lentivirus, adeno-associated virus (AAV), and adenovirus, or a bocavirus.

In certain embodiments, the expression control system is activated by a tetracycline class antibiotic. In certain embodiments, the tetracycline class antibiotic is doxycycline.

### Methods of Treatment

Provided herein are methods of treatment of damage to the nervous system after injury or disease. The methods include administering of an effective amount of DOX-activatable glial cells to a subject in need thereof and administering an effective amount of DOX at a desirable time. Administration of the DOX activates the response of the cells and induces differentiation of the cells in vivo.

In certain embodiments, the patient has damage to the brain and/or the spinal cord. In some embodiments, treatment using the method provided herein improves motor recovery of the patient when compared to an untreated control. In certain embodiments, the cells display anatomical and/or functional evidence of connectivity. In certain embodiments, treatment further comprises at least one additional administration of DOX to the subject.

In certain embodiments, the method provided herein further comprises patient participation in rehabilitative physical therapy. In certain embodiments, the patient is treated after surgical treatment of the nervous system.

### Cell Administration

The DOX-activable cells described herein may be delivered to a subject by any means. The compositions described herein can be formulated for intravenous (IV), intramuscular (IM), intra-articular (IA) and intrathecal (lumbar puncture) administration. The compositions can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

These cells may be administered alone, as pharmaceutical composition in combination with diluents and/or carriers and/or buffers, and other components. In one embodiment, they may be administered in saline. In another embodiment, in a hydrogel. Among other formulations, both saline and hydrogel formulations are contemplated for delivery by injection. Other components may include cytokines, cells, or other agents conventionally used to repair a damaged nervous system. Compositions may include stabilizers, antioxidants, and/or preservatives. Compositions may include, e.g., neutral buffered saline or phosphate buffered saline.

Carriers may include pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. Carriers also includes any and all coatings, antibacterial and antifungal agents, and absorption delaying agents, and the like that are compatible with the activity of the components, e.g., cells, to be delivered, and are physiologically acceptable to the patient. Supplementary active compounds may also be incorporated into the compositions. Pharmaceutically acceptable salt of the compound or molecule useful within the invention.

Other ingredients that may be included are excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Still other additional ingredients that may be included in the pharmaceutical compositions used in the practice of the invention and are known in the art and described elsewhere.

In certain embodiments, the cells described herein are delivered directly to the site of injury. In other embodiments, the cells are delivered via intrathecal administration. In other embodiments, the cells are delivered IV. In other embodiments, the cells are delivered intramuscularly or intra-arterially. The cells may be delivered in one administration, or in several administrations over a course of time.

Effective amounts of the modified cells and other aspects of a pharmaceutical composition may be determined by one of skill in the art, including by a physician. Such amounts may be determined by with consideration of the age and/or weight of a patient, and further by the size, condition, location, and/or severity of the wound or wounds to be treated. In one embodiment, administration of from 1 to 100 million cells is appropriate. Further ranges/subranges, and integers within those ranges/subranges, are also contemplated, including but not limited to from 1 to 10 million, 1 to 5 million, 1 to 1 million, 1 to 500,000, 1 to 400,000, 1 to 300,000, 1 to 250,000, 1 to 200,000, 1 to 150,000, 1 to 100,000, 1 to 50,000, 1 to 40,000, 1 to 30,000, 1 to 20,000, 1 to 10,000, 1 to 5,000, 1 to 2,500, 1 to 1,000, 1 to 100, 10 million to 100 million, 50 million to 100 million, 75 million to 100 million.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of alleviation or amelioration from one or more symptoms associated with a damaged nervous system.

The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

In certain embodiments, the cells described herein are provided with one or more additional therapies for the damaged nervous system. Treatment for includes medications such as those used to promote nerve cell regeneration or to improve the function of existing nerves.

In certain embodiments, the cells described herein are provided with one or more additional therapies for treating a damaged nervous system, including without limitation rehabilitative physical therapy.

Still further provided are kits comprising one of more of the cells described herein in one or more vials, tubes, or other suitable vessels. The kit may further comprise a syringe or other medical instrument suitable to deliver a composition to a subject.

### DOX Activation of Cells

Provided herein are cells that are responsive to DOX. In certain embodiments, the DOX is delivered in vivo. In certain embodiments, the DOX is delivered using oral, intravenous (IV), intramuscular (IM), intra-articular (IA) or intrathecal (lumbar puncture) administration.

The duration for delivery of the DOX is in an amount sufficient to activate the cells without damaging the surrounding tissues. In certain embodiments, the stimulus is provided at the same time that the cells are administered. In certain embodiments, the stimulus is delivered after administration of the cells. In certain embodiments, the stimulus is administered at least 1, 2, 3, 4, 5, 6, 7, 8, 9, 10, or more times after the initial stimulus. In certain embodiments, the stimulus is administered one or more times per hour for several hours, days, weeks, or months. In certain embodiments, the stimulus is administered one or more times per day for several days, weeks or months.

In certain embodiments, the cells will be stimulated, by DOX delivery, after the cells are administered to a patient. In certain embodiments, the DOX stimulus is delivered from 15-21 days after the cells are administered. Further ranges/subranges, and integers within those ranges/subranges, are also contemplated, including but not limited to at least about 1 day, 2 days, 3 days, 4 days, 5 days, 6 days, 7 days, 8 days, 9 days, 10 days, 11 days, 12 days, 13 days, 14 days, 1 week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, or 12 months after administration of the cells. In certain embodiments, the DOX is delivered with the cells.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### Examples

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: Transplanting Glial Progenitor Cells to Promote Repair of the Injured Cervical Cord

The injured adult mammalian spinal cord is not capable of self-repair. However, some treatments, especially those capable of recapitulating development, can promote repair and functional recovery after spinal cord injury (SCI). Developing glial progenitor cells (GRPs) have been shown to reduce scarring, promote axonal growth, and facilitate repair post-SCI.

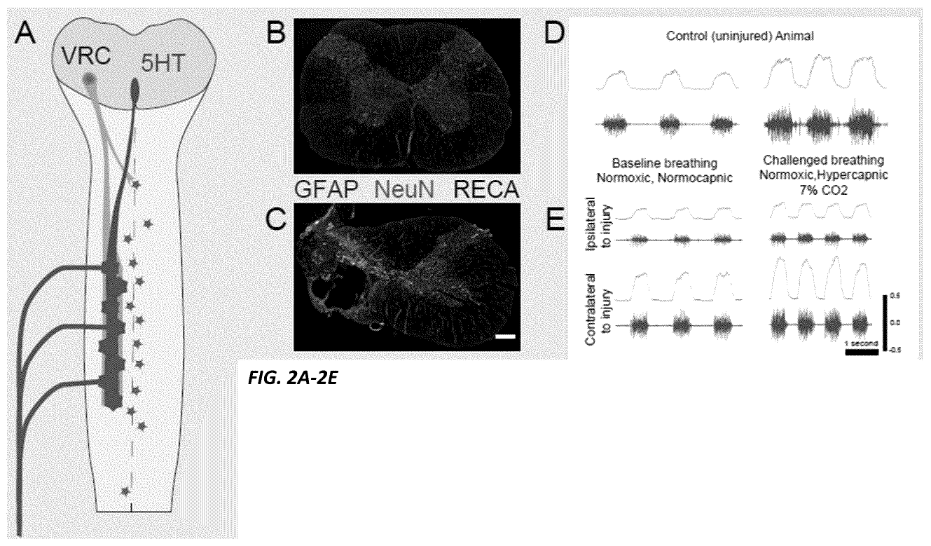

Building on this, GPRs were transplanted to an acutely and chronically injured spinal cord and observed for a month. FIG. 2 shows the effect of a cervical cord injury on an adult rat. The cross-sections throw the injured epicenter and compared to a healthy control reveals significant white and grey matter disruption and glial scarring. These injuries typically result in immediate respiratory arrest, followed by progressive recovery of voluntary breath 30-60 minutes posh injury.

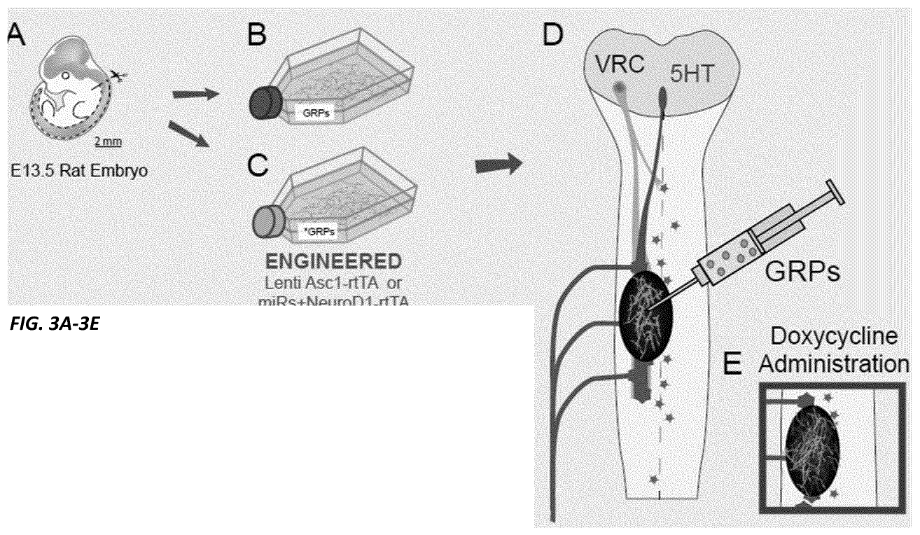

Glial cells were then obtained from a 13.5-day old rat embryo and transplanted into the lesion cavity acutely (1 week) or chronically (1 month) post SCI. A schematic of this procedure is shown in FIG. 3. The Glial cells were mechanically dissected out of a green fluorescent protein (GFP) expressing rat pup and enzymatically disassociated. These cells were cultured in two separate groups for three days to reach confluency. One of the groups was cultured with a lenti-virus to allow for DOX-induced gene regulation and direct reprogramming of the glial cells into neurons. The cells were then stereotaxically injected into the legion cavity of two different rat groups. The group administered the modified cells were then given doxycycline. RNA samples are collected before and after transplantation and anatomical tracing is used to map repair. Electrophysiology is also used to assess neural function and recovery from injury.

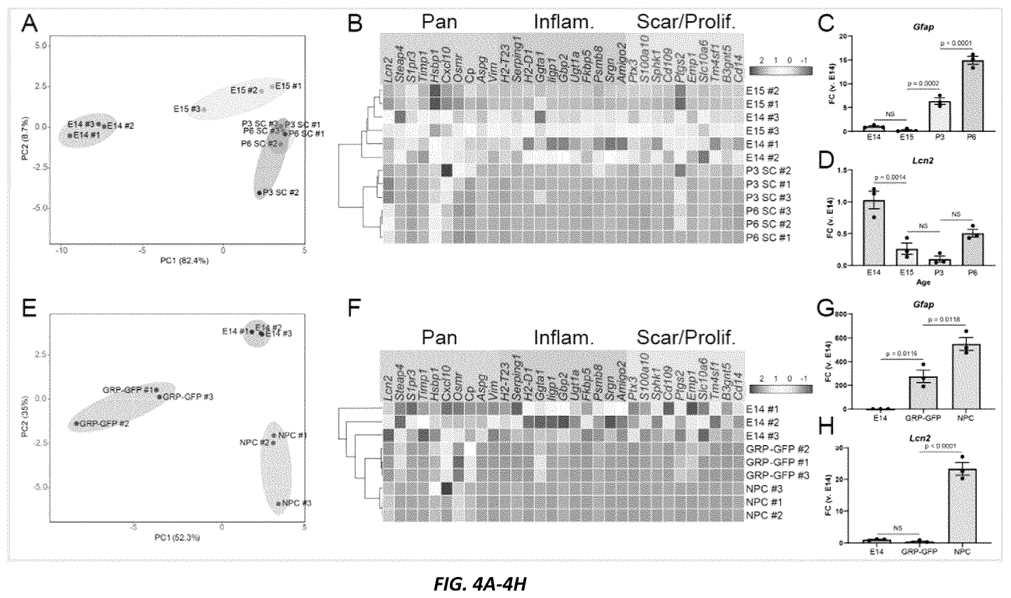

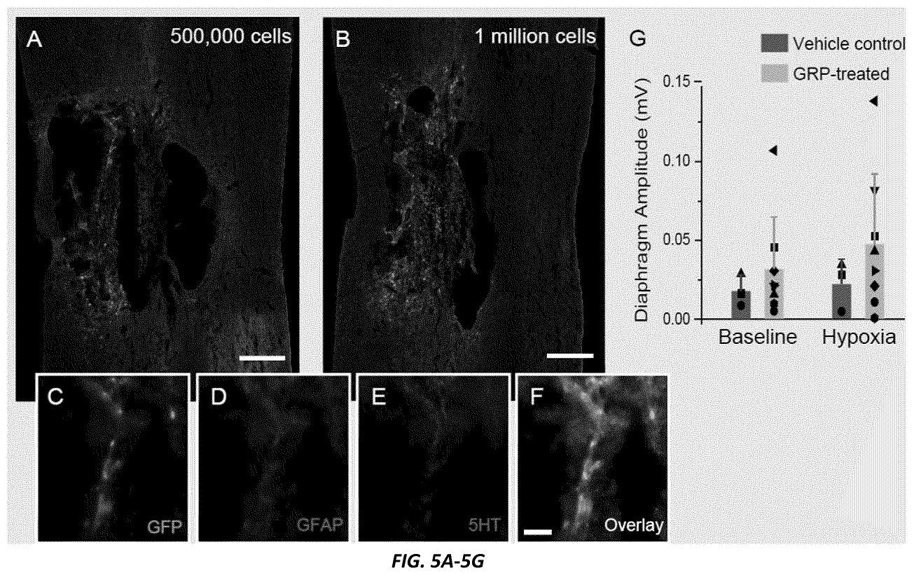

Th RNA isolated from these cells was then compared to developing spinal cords and demonstrated close clustering of biological replicates. A heatmap, provided in FIG. 4B, compares the expression of pan-astrocytic genes and genes relevant to either more “inflammatory” (Inflam.) and or “scar forming or proliferative” (Scar/Prolif.) glial phenotypes between embryonic (E14,15) and postnatal (P3,6) spinal cords. Specific examples of these genes are also provided in FIGS. 4C and 4D. Both the upregulation of Gfap (cytoskeletal maturation with development), and downregulation of Lcn2 (a gene for reactive, developing astrocytes) were expected with time. Similar analysis across the cultured cells (GRPs and NPCs, compared to E14 spinal cord) is represented as a heatmap in FIG. 4F with additional specific examples of genes in FIGS. 4G and 4H. While the upregulation of Gfap suggests cytoskeletal maturation in both NPCs and GRPs (G), the significantly greater Gfap and upregulated Len2 suggests that the astrocytes within NPCs are in a more “reactive” state that GRPs.

In the acute injury model, the glial cells were transplanted 1 week after injury at two doses: 500,000 cells/animal and 1 million cells/animal. Despite poor immunosuppression, some cell survival was evident in both groups, with 1 million cell-dose recipients resulting in greater anatomical repair (greater tissue continuity, decreased cavitation). The surviving cells expressed GFAP and guide sprouting 5HT axons into and through the injury site. Amplitude of the diaphragm contraction was measured with electromyography to assess recovery 4 weeks after transplantation (green bar, 500,000 cells/animal) and compared to vehicle-recipients (blue bar) under baseline and hypoxic challenge.

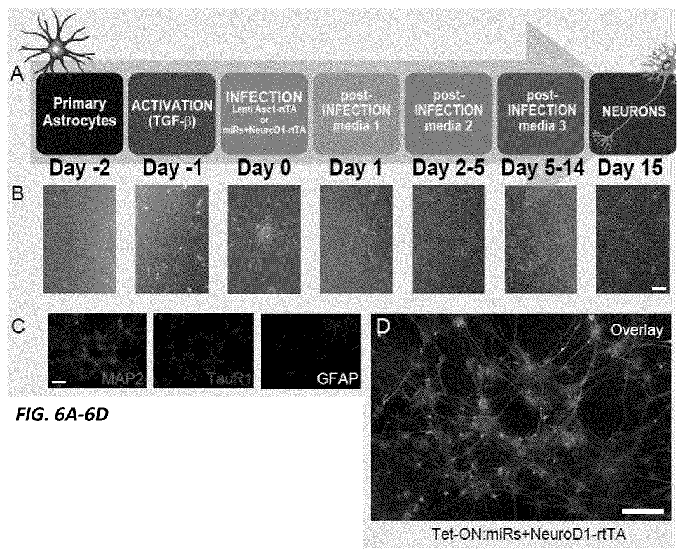

Lastly, direct cellular reprograming of the modified glial cells into neurons was performed by overexpressing neurogenic transcription factors (Ascl1 or a combination of miR124, miR9/9* and NeuroD1). Progressive transition from glial cells to neuron was shown in FIG. 6B. Immunocytochemistry confirmed the transition of glial cells to neurons.

Refining bankable and stable populations of glial restricted progenitors (GRPs) produces cells with both inflammatory and scarring/proliferating phenotypes but are less ‘reactive’ than other neural progenitor cells. GRPs transplanted into the injured spinal cord are shown to integrate with the injured tissue and support growth of serotonergic axons across the injury site. GRP recipients exhibit modest improvement in diaphragm activity 4 weeks after transplantation.

Additionally, Doxycycline-regulated transcription factor expression in glial cells results in effective conversion of astrocytes into defined populations of neurons.

### Example 2: Astrocyte to Neuron Reprogramming for Spinal Cord Repair

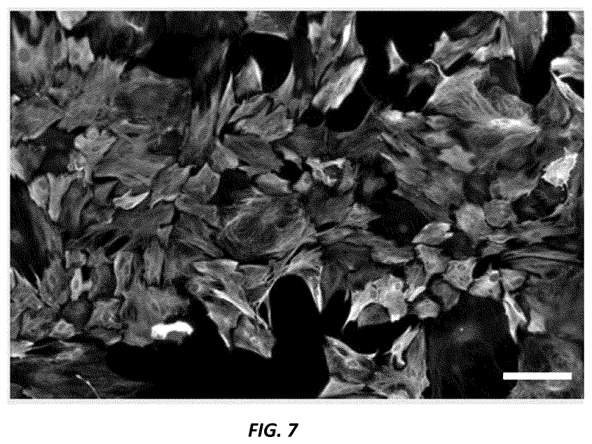

Despite some limited neuroplasticity, cells destroyed during SCI are incapable of spontaneous repair, leaving functional deficits permanent with no curative treatments. Instead of being destroyed, one cell type-astrocytes-increase in number and reactivity after SCI. Presented herein is the use genetic reprogramming methods to convert primary astrocyte-generated from postnatal days 2-3 rat cortex (FIG. 7)—to neurons, to promote repair and functional recovery.

The reprogramming strategy employed herein is the Tet-on system, which uses a reverse tetracycline-controlled transactivator—rtTA—to promote transcription and induce the expression of certain genes, which determine the fate of the cell as it matures.

Two conditions play important roles in neuronal differentiation. Leveraging these conditions using activation and expression facilitates the reprogramming from astrocytes to neurons. The first condition is Ascl1; a transcription factor (TF) that binds to DNA and promotes the transcription of neuronal gene expression. The second condition is a combination of the microRNAs miR124 and mi R9/9* (identified hereafter as “the miRNAs”) which contribute to neuronal phenotype.

3 experimental conditions were used to generate the data herein: (1) rtTA (control), (2) rtTA+Ascl1, (3) rtTA+miRs. Additionally, a cocktail of molecules was added to facilitate reprogramming and neuronal maturation:
Doxycycline—tetracycline antibiotic, activates Tet-on system and induces the transcription
bFGF—cell proliferation growth factor,
BDNF—brain derived neurotrophic factor,
GDNF—glial cell derived neurotrophic factor,
Forskolin—increases intracellular levels of cAMP, and
Ascorbic acid—acts as an antioxidant.

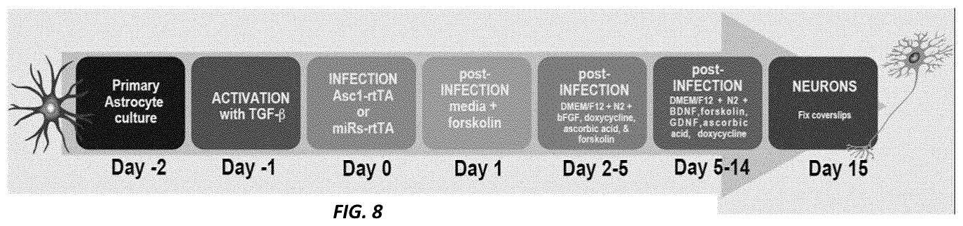

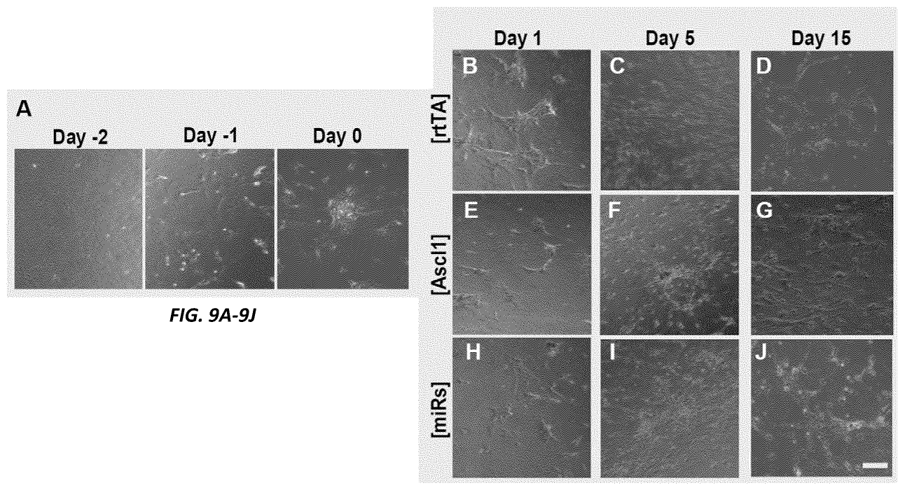

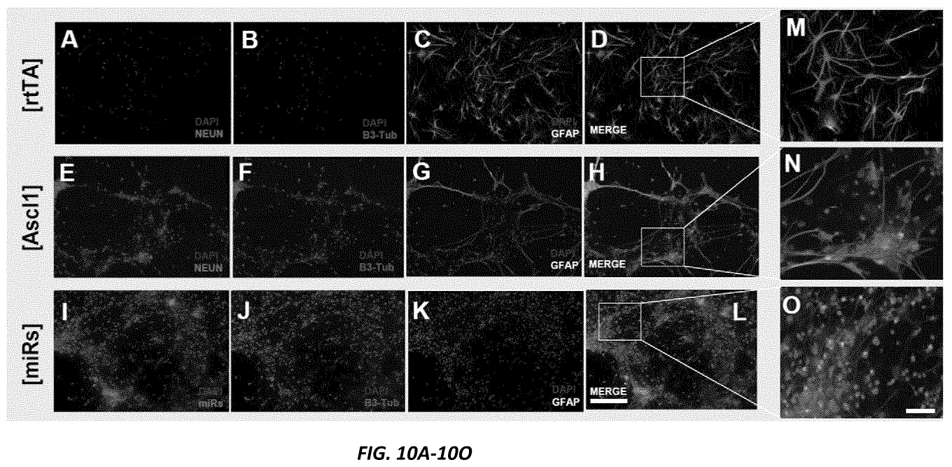

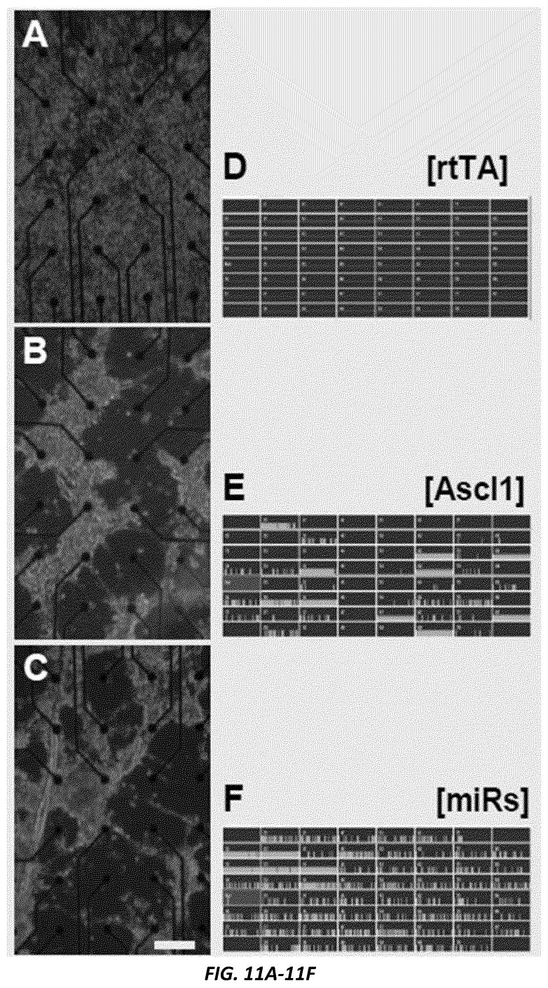

A timeline of the conversion of primary astrocytes to neurons is shown in FIG. 8. At day 0, the cells were plated in one of the three conditions indicated above. Several phase images of the cells were taken of the cells from days −2-15 to investigate the changes in cell morphology. Prior to introduction to these conditions, the cells displayed an undifferentiated astrocytic morphology. At day 1, the rtTA, Ascl1, miRs all promoted elongation of processes, but the greatest were in those treated with Ascl1 and miRs. At day 5, each condition showed further elongation and a large increase in the number oc cells/nuclei. Ascl1 and miRs had clearer neural processes and little visible astrocytic morphology, while rtTA had more astrocytic characteristics. After 15 days, the Ascl1 and miRs showed an increased neuronal morphology (thin neurites) but the rtTA maintained thicker processes and star-shaped cells.

An immunocytochemical analysis was performed for astrocytic marker GFAP, neuronal nuclear protein NeuN and neuronal cytoskeletal marker β3-tubulin to assess in vitro protein expression to confirm conversion from astrocytes to neurons. rtTA was positive for GFAP but lacked staining indications for NeuN and β3-tubulin. This result indicates that the cells exposed to rtTA were not converted to neurons. Conversely, both Ascl1 and miR treated cells showed low positivity for GFAP and high positivity for NeuN and β3-tubulin labeling, thereby supporting the conversion of these cells to a high neuron population. Although not all cells were converted, this is beneficial because the remaining glial cells support the survival of the new neurons.

Next, the activity of the treated cells was analyzed for neuron-like behavior. Cells treated with rtTA, Ascl, and miRs were all analyzed for neuronal spikes and neural bursts. The absence of neuronal spikes in cells treated with rtTA indicated that there was no astrocyte-to-neuron conversion. Conversely, the cells treated with Ascl1 or miRs had significant spikes and increased neuronal bursts indicating that astrocyte-to-neuron conversion was successful.

The data provided herein indicates that Ascl1 and miRs can be used to reprogram brain astrocytes to neurons. Morphology, protein expression, and electrical activity indicated that astrocyte to neuron conversion is successful in vitro.

Each and every patent, patent application, and publication, including publications listed herein and publicly available nucleic acid and amino acid sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

What is claimed is:

1. An engineered glial cell comprising an exogenous nucleic acid comprising a transcription factor under the control of a regulatable promoter.

2. The engineered glial cell according to claim 1, wherein the regulatable promoter is a doxycycline (DOX)-inducible expression control system.

3. The engineered glial cell of claim 1, wherein the transcription factor comprises one or more of VSX2 (also called HOX10), Ascl1, miR 124, and miR9/9*.

4. A method for repairing a damaged nervous system in a subject in need thereof, the method comprising administering to the subject the cells of claim 1 and inducing expression of said transcription factor in said cells, thereby inducing reprogramming of the cells into neurons.

5. The method of claim 4, wherein the damage to the nervous system is caused by injury or disease.

6. The method of claim 4, wherein said inducing expression is by administration of DOX to the subject.

7. The method of claim 4, wherein inducing expression in the cells is weeks or months after the cells have been administered.

8. The method of claim 4, wherein the patient has damage to at least one of the brain or the spinal cord.

9. The method of claim 4, wherein unmodified cells are obtained from the subject and used to generate the engineered glial cells.

10. The method of claim 4, wherein unmodified cells are obtained from a donor and used to generate the engineered glial cells.

11. The method of claim 9, wherein the unmodified cells are human or non-human PCS.

12. The method of claim 4, wherein administration of the engineered cells improves motor recovery of the patient when compared to an untreated control.

13. The method of claim 4, wherein the engineered cells display at least one of anatomical and functional evidence of connectivity.

14. The method of claim 4, wherein administration of the engineered cells occurs prior to surgical treatment.

15. The method of claim 4, wherein administration of the engineered cells occurs after surgical treatment.

16. The method of claim 4, further comprising said patient participating in rehabilitative physical therapy.

17. The method of claim 16, wherein said rehabilitative physical therapy maintains or strengthens muscle function or redevelops fine motor skills.
